(19) United States
(12) Patent Application Publication
(10) Pub. No.: US 2012/0093939 A1
(43) Pub. Date: Apr. 19, 2012

(54) ORAL FORMULATIONS

(75) Inventors: Adam Payne, Oklahoma City, OK (US); Dennis Hair, Oklahoma City, OK (US); Richard Harty, Oklahoma City, OK (US); Shoufeng Li, Oklahoma City, OK (US); Sachin Malhorta, Oklahoma City, OK (US)

(73) Assignee: ALTHEUS THERAPEUTICS, INC., Oklahoma City, OK (US)

(21) Appl. No.: 13/140,425

(22) PCT Filed: Dec. 16, 2009
(86) PCT No.: PCT/US09/68174

§ 371 Date: Dec. 6, 2011

Related U.S. Application Data
(60) Provisional application No. 61/138,506, filed on Dec. 17, 2008.

(51) Int. Cl. A61K 31/606; A61K 31/197; A61K 9/14

(52) U.S. Cl. 424/497; 514/166; 514/562

(57) ABSTRACT

Disclosed are pharmaceutical compositions comprising immediate release and sustained release formulations of 5-aminosalicylic acid, or a pharmaceutically acceptable salt or ester thereof, and/or N-acetylcysteine, or a pharmaceutically acceptable salt or ester thereof, for release in the lower gastrointestinal tract.

### RELATED APPLICATIONS

The present application claims priority to the U.S. Provisional Application Ser. No. 61/138,506, filed on Dec. 17, 2008, by Harty et al., and entitled “ORAL FORMULATIONS,” the entire disclosure of which is incorporated herein by reference.

### FIELD OF THE INVENTION

The present invention is in the field of pharmaceutical compositions, and particularly in the field of oral formulations that release the pharmaceutically active ingredient in the lower gastrointestinal tract.

### BACKGROUND OF THE DISCLOSURE

Certain gastrointestinal diseases, such as inflammatory bowel disease (IBD), afflict the lower gastrointestinal (GI) tract. For example, ulcerative colitis specifically affects the colon, while Crohn's Disease mainly affects the lower ileum and/or the colon.

It has been known in the art that certain anti-inflammatory drugs, both steroidal and non-steroidal, are useful in the treatment of the active disease and in maintaining remission when the activity of the disease has been reduced. Some of the better-known drugs for the treatment of IBD include the derivatives of salicylic acids, such as 5-aminosalicylic acid (the active ingredient in Mesalamine and Mesalazine), 4-aminosalicylic acid, and steroids such as prednisone and budesonide (marketed under the trade name ENTOCORT®).

It is desirable to have the therapeutic drugs for the treatment of IBD to be released in or near the lower gastrointestinal tract. These drugs are most effective when they act topically in the diseased area. If the drugs are released further upstream in the GI tract, they get absorbed in the blood stream. Higher systemic concentrations of these drugs might reduce their therapeutic effect while increasing the incidents of their adverse side effects. Formulations are used to target the release of the therapeutic agents in the diseased area.

### SUMMARY OF THE INVENTION

Disclosed herein are pharmaceutical compositions comprising a plurality of first pellets each comprising i) a core comprising 5-aminosalicylic acid, or a pharmaceutically acceptable salt or ester thereof, and a first polymer; and ii) an enteric coating;

In some embodiments, the pharmaceutical compositions further comprise a plurality of second pellets each comprising i) a core comprising N-acetylcysteine, or a pharmaceutically acceptable salt or ester thereof, and a first polymer; and ii) an enteric coating.

In some embodiments, the pharmaceutical compositions further comprise a plurality of third pellets each comprising i) a core comprising 5-aminosalicylic acid, or a pharmaceutically acceptable salt or ester thereof, and a first polymer; ii) a first enteric coating; and iii) a second enteric coating.

In some embodiments, the pharmaceutical compositions further comprise a plurality of fourth pellets each comprising i) a core comprising N-acetylcysteine, or a pharmaceutically acceptable salt or ester thereof, and a first polymer; ii) a first enteric coating; and iii) a second enteric coating.

Also disclosed herein are pharmaceutical compositions comprising one of the following combinations of the above pellets: a) a plurality of the first pellets and a plurality of the second pellets; b) a plurality of the first pellets and a plurality of the third pellets; c) a plurality of the first pellets and a plurality of the fourth pellets; d) a plurality of the second pellets and a plurality of the third pellets; e) a plurality of the second pellets and a plurality of the fourth pellets; f) a plurality of the third pellets and a plurality of the fourth pellets; g) a plurality of the first pellets, a plurality of the second pellets, and a plurality of the third pellets; h) a plurality of the first pellets, a plurality of the second pellets, and a plurality of the fourth pellets; i) a plurality of the first pellets, a plurality of the third pellets, and a plurality of the fourth pellets; j) a plurality of the second pellets, a plurality of the third pellets, and a plurality of the fourth pellets; and k) a plurality of the first pellets, a plurality of the second pellets, a plurality of the third pellets, and a plurality of the fourth pellets.

### DETAILED DESCRIPTION OF THE EMBODIMENTS

The present inventors have discovered that administering a non-steroidal anti-inflammatory compound, such as 5-aminosalicylic acid (5-ASA), or a pharmaceutically acceptable salt or ester thereof, in combination with an antioxidant, such as N-acetylcysteine (NAC), or a pharmaceutically acceptable salt or ester thereof, has unexpected and synergistic effects in the treatment of IBD. In addition, the present inventors have discovered that administering an antioxidant, such as N-acetylcysteine, or a pharmaceutically acceptable salt or ester thereof, by itself or as an adjunct therapy has synergistic effect in increasing the therapeutic effect of mainline therapy.

It is discovered that it is best for these therapeutic compounds to be released in the lower GI tract at or near the diseased areas. Because the diseased areas, especially in Crohn's disease, can affect the entire length of the small intestine, including upper parts of the jejunum all the way through the lower parts of the ileum, the ileocecal region, and the colon, formulations are disclosed herein that provide for an immediate release of the compounds following the clearance from the stomach, in addition to a more delayed, or sustained, release of the compounds that allow for the release of the compounds in the lower GI tract.

Thus, in the first aspect, disclosed herein are a plurality of first pellets each comprising:
a) a core comprising 5-aminosalicylic acid, or a pharmaceutically acceptable salt or ester thereof, and a first polymer; and
b) a first enteric coating substantially insoluble in gastric juice.

By “substantially insoluble” it is meant that less than 10% by weight of the enteric coat has dissolved after exposure to the solution for one hour. Thus, a polymer or enteric coating that is substantially insoluble in gastric juice will dissolve less than 10% by weight after being exposed for one hour to an aqueous solution having a pH of less than 2. It is understood that some polymers disintegrate in aqueous solutions. This disintegration is not the same as dissolving. For a compound or polymer to be soluble, there needs to be a concentration of the compound or polymer in the solvent having solute-solvent interactions, as understood in the chemical arts.

The term “pharmaceutically acceptable salt” refers to a formulation of a compound that does not abrogate the biological activity and properties of the compound. Pharmaceutical salts can be obtained by reacting a compound of the invention with inorganic acids such as hydrochloric acid, hydrobromic acid, sulfuric acid, nitric acid, phosphoric acid, methanesulfonic acid, ethanesulfonic acid, p-toluenesulfonic acid, salicylic acid and the like. Pharmaceutical salts can also be obtained by reacting a compound of the invention with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like.

The term “ester” refers to a chemical moiety with formula —(R)ₙ—COOR′, where R and R′ are independently selected from the group consisting of alkyl, cycloalkyl, aryl, heteroaryl (bonded through a ring carbon) and heteroalicyclic (bonded through a ring carbon), and where n is 0 or 1.

In some embodiments, the 5-aminosalicylic acid, or a pharmaceutically acceptable salt or ester thereof, of the plurality of first pellets is present in between about 10% to about 90% by weight. Throughout this disclosure the term “by weight” refers to the weight of the pellet, i.e., the core and the enteric coating that coats the pellet, or the core and the sustained release coating and the enteric coating that coat the pellet.

In other embodiments, the 5-aminosalicylic acid, or a pharmaceutically acceptable salt or ester thereof, of the plurality of first pellets is present in between about 30% to about 90% by weight, or between about 50% to about 90% by weight, or between about 60% to about 90% by weight, or between about 60% to about 80% by weight, or between about 70% to about 80% by weight. In some embodiments, the 5-aminosalicylic acid, or a pharmaceutically acceptable salt or ester thereof, is present at about 65%. In some embodiments, the 5-aminosalicylic acid, or a pharmaceutically acceptable salt or ester thereof, is present at about 70%. In some embodiments, the 5-aminosalicylic acid, or a pharmaceutically acceptable salt or ester thereof, is present at about 75%.

Throughout the present disclosure the term “about” a certain value means that a range of value±10%, and preferably a range of value±5%, is contemplated. Thus, for example, having 70% 5-aminosalicylic acid includes 5-aminosalicylic acid being present between 63% and 87%, and preferably between 66.5% and 73.5%; or by way of another example, “about 100 mg” means that the contemplated value is between 90 mg and 110 mg, and preferably between 95 mg and 105 mg.

In some embodiments, the first polymer of the plurality of first pellets swells in aqueous media. In some embodiments, the first polymer of the plurality of first pellets forms a hydrogel in aqueous media. In certain embodiments, the first polymer of the plurality of first pellets is a cross-linked polymer. In some of these embodiments, the first polymer of the plurality of first pellets is selected from the group consisting of polyvinyl polypyrrolidone (PVPP), polyvinyl pyrrolidone (PVP), hydroxypropyl methylcellulose phthalate (HPMCP), hypromellose acetate succinate (HPMCAS), ethylcellulose, croscamolluse sodium (CMS-Na), Starch 1500, carboxymethyl cellulose sodium (CMC-Na), methacrylic acid copolymer, methyl methacrylate copolymer, and a polymer comprising methacrylic acid-methyl methacrylate copolymer. In further embodiments, the polymer is EUDRAGIT® L100 or EUDRAGIT® L30D 55. EUDRAGIT® polymers are well-known in the pharmaceutical industry. They are marketed by Pharma Polymers, a division of Evonik Industries, Darmstadt, Germany. In some embodiments, the polymer is PVPP XL-10.

In some embodiments, the first polymer of the plurality of first pellets is present in between about 0.1% to about 50% by weight. In other embodiments, the first polymer of the plurality of first pellets is present in between about 1% to about 40% by weight, or between about 1% to about 30% by weight, or between about 1% to about 10% by weight, or between about 3% to about 8% by weight, or between about 5% to about 7% by weight. In some embodiments, the first polymer is present in about 4.2%. In some embodiments, the first polymer is present in about 5.4%. In some embodiments, the first polymer is present in about 6.6%.

In some embodiments, the core of the plurality of first pellets further comprises a second polymer. In some of these embodiments, the second polymer is a polymeric sugar. Polymeric sugars, or polysaccharides, include those sugars that are linked together through α-1,4 glycosidic bonds or β-1,4 glycosidic bonds. Examples of polymeric sugars include starch, glycogen, and cellulose. In some embodiments, the second polymer is cellulose. In some of these embodiments, the cellulose is derivatized, e.g., alkylated. In some embodiments, the cellulose is microcrystalline cellulose (PH101).

In some embodiments, the second polymer is present in between about 0.1% to about 50% by weight. In other embodiments, the second polymer is present in between about 1% to about 40% by weight, or between about 5% to about 30% by weight, or between about 5% to about 20% by weight, or between about 5% to about 15% by weight, or between about 8% to about 13% by weight. In some embodiments, the second polymer is present in about 11%. In some embodiments, the second polymer is present in about 14%. In some embodiments, the second polymer is present in about 18%. In some embodiments, the second polymer is present in about 9%.

In some embodiments, the core of the plurality of first pellets is made by passing 5-ASA, and the plurality of polymers (i.e., first polymers and second polymers, if present) through screening sieves; whereupon they are weighed out into high shear granulator and mixed; a wetting agent is added to the mixture of 5-ASA and first polymers; the wet mixture is granulated; the granulated wet mass is then extruded using an extrusion hole; and the extruded material is spheronized and oven dried. In some embodiments the wetting agent used is water.

In some embodiments, the core of the plurality of first pellets comprises a barrier coating before the enteric coating is added. In some embodiments the barrier can be a solution of a mixture of polymers HPMC and PEG 400. In some embodiments, the mixture of barrier polymers is at about 1:1 weight ratio. In some embodiments, the barrier coating is applied in a fluid bed using a process similar to the enteric coating procedure described below.

In some embodiments, the first enteric coating of the plurality of first pellets is substantially insoluble in media with pH<3. In other embodiments, the first enteric coating of the plurality of first pellets is substantially insoluble in media with pH<4, or pH<5, or pH<6. In some embodiments, the first enteric coating of the plurality of first pellets is substantially insoluble in media with pH<6.8.

In some embodiments, the first enteric coating of the plurality of first pellets comprises a polymer insoluble in gastric juice. In some of these embodiments, the polymer comprises methacrylic acid copolymer, methyl methacrylate copolymer, and a polymer comprising methacrylic acid-methyl methacrylate copolymer. In some embodiments, the first enteric coating comprises EUDRAGIT® S100.

In some embodiments, the polymer is present in between about 0.1% to about 50% by weight. In other embodiments, the polymer is present in between about 1% to about 40% by weight, or between about 1% to about 30% by weight, or between about 1% to about 10% by weight, or between about 3% to about 9% by weight, or between about 6% to about 8% by weight. In some embodiments, the polymer is present in about 8.8%. In some embodiments, the polymer is present in about 6.2%. In some embodiments, the polymer is present in about 11.9%.

In some embodiments, the first enteric coating further comprises a pharmaceutically acceptable plasticizer. In some of these embodiments, the plasticizer is an alkyl citrate. In certain embodiments, the alkyl citrate is selected from the group consisting of triethyl citrate (TEC), acetyl triethyl citrate (ATEC), tributyl citrate (TBC), acetyl tributyl citrate (ATBC), trioctyl citrate (TOC), acetyl trioctyl citrate (ATOC), trihexyl citrate (THC), acetyl trihexyl citrate (ATHC), butyryl trihexyl citrate (BTHC, trihexyl o-butyryl citrate), and trimethyl citrate (TMC). In some embodiments, the plasticizer is triethyl citrate (TEC).

In some embodiments, the plasticizer is present in between about 0.1% to about 50% by weight. In other embodiments the plasticizer is present in between about 0.1% to about 20% by weight, or between about 0.1% to about 10% by weight, or between about 0.1% to about 1% by weight, or between about 0.3% to about 0.9% by weight, or between about 0.6% to about 0.8% by weight. In some embodiments, the plasticizer is present in about 0.08%. In some embodiments, the plasticizer is present in about 0.28%. In some embodiments, the plasticizer is present in about 0.58%. In some embodiments, the plasticizer is present in about 0.88%.

In some embodiments, the first enteric coating further comprises an inert mineral. An inert mineral is a mineral, i.e., an inorganic compound or salt, that is pharmaceutically acceptable and does not interfere with the pharmacological action of the therapeutic compound. In some embodiments, the inert mineral is a mineral of magnesium. In other embodiments, the mineral of magnesium is magnesium silicate. In certain embodiments, the first enteric coating further comprises talc.

In some embodiments, the inert mineral is present in between about 0.1% to about 50% by weight. In other embodiments, the inert mineral is present in between about 0.1% to about 40% by weight, or between about 0.5% to about 30% by weight, or between about 0.5% to about 10% by weight, or between about 0.5% to about 2% by weight, or between about 0.8% to about 1.5% by weight. In some embodiments, the inert mineral is present in about 1.7%. In some embodiments, the inert mineral is present in about 3.6%. In some embodiments, the inert mineral is present in about 11.0%.

In some embodiments, the enteric coating of the plurality of first pellets is prepared by dispersing triethyl citrate in a wetting agent; talc is added to the triethyl citrate dispersion; the dispersion is homogenized; Eudragit S 100 is added slowly to the talc/triethyl citrate dispersion while stirring to maintain a uniformly dispersed enteric matrix before coating it on the core of the plurality of first pellets as described below.

In some embodiments, the core of the first pellets or the core of the barrier coated first pellets are enteric coated in a fluid bed using bottom-spray technique with a spray nozzle.

In some embodiments, the plurality of enteric coated cores of the first pellets is cured in a fluid bed. In other embodiments, the pellets are cured in an oven; using SiO₂ to prevent pellet adhesion in the oven.

In another aspect, disclosed herein are a plurality of second pellets each comprising:
a) a core comprising N-acetylcysteine, or a pharmaceutically acceptable salt or ester thereof, and a first polymer; and
b) a second enteric coating substantially insoluble in gastric juice.

In some embodiments, the N-acetylcysteine, or a pharmaceutically acceptable salt or ester thereof, in the plurality of second pellets is present in between about 10% to about 90% by weight. In some embodiments, the N-acetylcysteine, or a pharmaceutically acceptable salt or ester thereof, is present in between about 30% to about 90% by weight, or between about 50% to about 90% by weight, or between about 60% to about 90% by weight, or between about 60% to about 80% by weight, or between about 70% to about 80% by weight. In some embodiments, the N-acetylcysteine, or a pharmaceutically acceptable salt or ester thereof, is present in about 51%. In some embodiments, the N-acetylcysteine, or a pharmaceutically acceptable salt or ester thereof, is present in about 57%. In some embodiments, the N-acetylcysteine, or a pharmaceutically acceptable salt or ester thereof, is present in about 67%. In some embodiments, the N-acetylcysteine, or a pharmaceutically acceptable salt or ester thereof, is present in about 77%.

In some embodiments, the first polymer of the plurality of second pellets swells in aqueous media. In some embodiments, the first polymer of the plurality of second pellets forms a hydrogel in aqueous media. In some of these embodiments, the first polymer of the plurality of second pellets is a cross-linked polymer. In certain embodiments, the first polymer of the plurality of second pellets is selected from the group consisting of polyvinyl polypyrrolidone (PVPP), polyvinyl pyrrolidone (PVP), hydroxypropyl methylcellulose phthalate (HPMCP), hypromellose acetate succinate (HPMCAS), ethylcellulose, croscamolluse sodium (CMS-Na), Starch 1500, methacrylic acid copolymer, methyl methacrylate copolymer, and a polymer comprising methacrylic acid-methyl methacrylate copolymer. In some embodiments, the polymer is EUDRAGIT® L100 or EUDRAGIT® L30D 55. In certain embodiments, the polymer is PVPP-XL 10.

In some embodiments, the first polymer of the plurality of second pellets is present in between about 0.1% to about 50% by weight. In certain embodiments, the first polymer of the plurality of second pellets is present in between about 1% to about 40% by weight, or between about 1% to about 30% by weight, or between about 1% to about 10% by weight, or between about 3% to about 8% by weight, or between about 3% to about 6% by weight. In some embodiments, the first polymer is present in about 0.8%. In some embodiments, the first polymer is present in about 1.6%. In some embodiments, the first polymer is present in about 3.8%. In some embodiments, the first polymer is present in about 6.6%.

In some embodiments, the core of the plurality of second pellets further comprises a second polymer. In certain embodiments, the second polymer is a polymeric sugar, as defined above. In certain embodiments, the second polymer is cellulose, which can be microcrystalline cellulose.

In some embodiments, the second polymer is present in between about 0.1% to about 50% by weight. In certain embodiments, the second polymer is present in between about 1% to about 40% by weight, or between about 5% to about 30% by weight, or between about 5% to about 20% by weight, or between about 5% to about 15% by weight, or between about 8% to about 15% by weight. In some embodiments, the second polymer is present in about 2.9%. In some embodiments, the second polymer is present in about 4.9%. In some embodiments, the second polymer is present in about 5.9%.

In some embodiments, the core of the plurality of second pellets further comprises a third polymer. In certain embodiments, the third polymer is a polymeric sugar, as defined above. In certain embodiments, the third polymer is cellulose, which can be ethylene cellulose (EC100 cps) or HPMC (K4M). In certain embodiments, the third polymer is polyvinyl pyrilodone, which can be K-29/32, S-630/32 or S-630.

In some embodiments, the third polymer is present in between about 0.1% to about 50% by weight. In certain embodiments, the third polymer is present in between about 1% to about 40% by weight, or between about 5% to about 30% by weight, or between about 5% to about 20% by weight, or between about 5% to about 15% by weight, or between about 8% to about 15% by weight.

In some embodiments, the core of the plurality of second pellets further comprises an inert matrix forming material. In some embodiments, the inert matrix forming material is selected from the group consisting of amphiphilic materials with high melting point. In certain embodiments, the inert matrix forming material is glyceryl behenate (Compritol 888ATO).

In some embodiments, the inert matrix forming material is present in between about 0.1% to about 50% by weight. In certain embodiments, the inert matrix forming material is present in between about 1% to about 40% by weight, or between about 5% to about 30% by weight, or between about 5% to about 20% by weight, or between about 5% to about 15% by weight, or between about 8% to about 15% by weight. In some embodiments, the inert matrix forming material is present in about 8.7%. In some embodiments, the inert matrix forming material is present in about 11.5%. In some embodiments, the inert matrix forming material is present in about 16.2%. In some embodiments, the inert matrix forming material is present in about 19.4%.

In some embodiments, the core of the plurality of second pellets further comprises an antioxidant. In certain embodiments, the antioxidant is selected from the group consisting of ascorbic acid, propyl gallate, tocopherols, tertiary butylhydroquinone, butylated hydroxyanisole and butylated hydroxytoluene. In some of these embodiments, the antioxidant is ascorbic acid.

In some embodiments, the antioxidant is present in between about 0.1% to about 50% by weight. In some of these embodiments, the antioxidant is present in between about 0.1% to about 20% by weight, or between about 0.1% to about 10% by weight, or between about 0.1% to about 1% by weight, or between about 0.1% to about 0.6% by weight, or between about 0.1% to about 0.4% by weight. In some embodiments, the antioxidant is present in about 0.56%. In some embodiments, the antioxidant is present in about 0.64%. In some embodiments, the antioxidant is present in about 0.96%. In some embodiments, the antioxidant is present in about 1.2%.

In some embodiments, the core of the plurality of second pellets is made by passing NAC, the first polymer, the second polymer, the inert matrix material and the antioxidant through screening sieves; whereupon they are weighed out into a high shear granulator and mixed; a wetting agent is added to the mixture of of NAC, the first polymer, the second polymer, the inert matrix material and the antioxidant; the wet mixture is granulated; the granulated wet mass is then extruded using an extrusion hole; and the extruded material is spheronized and oven dried. In some embodiments the wetting agent used is isporopyl alcohol. In certain embodiments the wetting agent used is water.

In some embodiments, the core of the plurality of first pellets further comprises a barrier coating before the enteric coating is added. In some cases the barrier comprises a mixture of the two polymers HPMC (E6) and PEG 400. In some embodiments, the mixture of barrier polymers is at 1:1 weight ratio. In some embodiments, the mixture of barrier polymers is at 1:10 weight ratio. In some embodiments, the barrier coating further comprises magnesium stearate. In some embodiments, the barrier coating is applied in a fluid bed using a process similar to the enteric coating procedure described below.

In some embodiments, the second enteric coating of the plurality of second pellets is substantially insoluble in media with pH<3. In other embodiments, the enteric coating of the plurality of second pellets is substantially insoluble in media with pH<4, or pH<5, or pH<6. In some embodiments, the enteric coating of the plurality of second pellets is substantially insoluble in media with pH<6.8.

In some embodiments, the second enteric coating of the plurality of second pellets comprises a polymer insoluble in gastric juice. In certain embodiments, the polymer comprises methacrylic acid copolymer, methyl methacrylate copolymer, and a polymer comprising methacrylic acid-methyl methacrylate copolymer. In some embodiments, the second enteric coating comprises EUDRAGIT® S100.

In some embodiments, the polymer is present in between about 0.1% to about 50% by weight. In some of these embodiments, the polymer is present in between about 1% to about 40% by weight, or between about 1% to about 30% by weight, or between about 1% to about 10% by weight, or between about 3% to about 10% by weight, or between about 7% to about 10% by weight. In some embodiments, the polymer is present in about 11.5%. In some embodiments, the polymer is present in about 14.5%. In some embodiments, the polymer is present in about 19.5%.

In some embodiments, the second enteric coating further comprises a pharmaceutically acceptable plasticizer. In some of these embodiments, the plasticizer is an alkyl citrate. In certain embodiments, the alkyl citrate is selected from the group consisting of triethyl citrate (TEC), acetyl triethyl citrate (ATEC), tributyl citrate (TBC), acetyl tributyl citrate (ATBC), trioctyl citrate (TOC), acetyl trioctyl citrate (ATOC), trihexyl citrate (THC), acetyl trihexyl citrate (ATHC), butyryl trihexyl citrate (BTHC, trihexyl o-butyryl citrate), and trimethyl citrate (TMC). In some embodiments, the plasticizer is triethyl citrate (TEC).

In some embodiments, the plasticizer is present in between about 0.1% to about 50% by weight. In certain embodiments, the plasticizer is present in between about 0.1% to about 20% by weight, or between about 0.1% to about 10% by weight, or between about 0.1% to about 1% by weight, or between about 0.3% to about 1% by weight, or between about 0.6% to about 1% by weight. In some embodiments, the plasticizer is present in about 1.4%. In some embodiments, the plasticizer is present in about 1.8%. In some embodiments, the plasticizer is present in about 2.0%.

In some embodiments, the second enteric coating further comprises an inert mineral. In some of these embodiments, the inert mineral is a mineral of magnesium. In certain embodiments, the mineral of magnesium is magnesium silicate. In some embodiments, the second enteric coating further comprises talc.

In some embodiments, the inert mineral is present in between about 0.1% to about 50% by weight. In some of these embodiments, the inert mineral is present in between about 0.1% to about 40% by weight, or between about 0.5% to about 30% by weight, or between about 0.5% to about 10% by weight, or between about 0.5% to about 2% by weight, or between about 0.8% to about 2% by weight. In some embodiments, the inert mineral is present in between about 0.9%. In some embodiments, the inert mineral is present in between about 1.9%. In some embodiments, the inert mineral is present in between about 2.9%.

In some embodiments, the enteric matrix of the plurality of second pellets is prepared by dispersing triethyl citrate in a wetting agent; talc is added to the triethyl citrate dispersion; the dispersion is homogenized; Eudragit S 100 is added slowly to the talc/triethyl citrate dispersion while stirring to maintain a uniformly dispersed enteric matrix before coating it on the core of the plurality of second pellets as described below.

In some embodiments, the core of the second pellets or the core of the barrier coated second pellets are enteric coated in a fluid bed using bottom-spray technique with a spray nozzle.

In some embodiments, the plurality of enteric coated cores of the second pellets is cured in a fluid bed. In other embodiments, the pellets are cured in an oven; using SiO₂ to prevent pellet adhesion in the oven.

In another aspect, disclosed herein are a plurality of third pellets each comprising:
a) a core comprising 5-aminosalicylic acid, or a pharmaceutically acceptable salt or ester thereof, and a first polymer;
b) a first sustained release coating substantially insoluble in gastric juice; and
c) a third enteric coating substantially insoluble in gastric juice.

In some embodiments, the 5-aminosalicylic acid, or a pharmaceutically acceptable salt or ester thereof, in the third pellets is present in between about 10% to about 90% by weight. In some of these embodiments, the 5-aminosalicylic acid, or a pharmaceutically acceptable salt or ester thereof, is present in between about 30% to about 90% by weight, or between about 50% to about 90% by weight, or between about 50% to about 80% by weight, or between about 50% to about 70% by weight, or between about 60% to about 70% by weight. In some embodiments, the 5-aminosalicylic acid, or a pharmaceutically acceptable salt or ester thereof, in the third pellets is present in about 64%. In some embodiments, the 5-aminosalicylic acid, or a pharmaceutically acceptable salt or ester thereof, in the third pellets is present in about 74%. In some embodiments, the 5-aminosalicylic acid, or a pharmaceutically acceptable salt or ester thereof, in the third pellets is present in about 84%.

In some embodiments, the first polymer of the plurality of third pellets swells in aqueous media. In some embodiments, the first polymer of the plurality of third pellets forms a hydrogel in aqueous media. In certain embodiments, the first polymer of the plurality of third pellets is a cross-linked polymer. In some of these embodiments, the first polymer of the plurality of third pellets is selected from the group consisting of polyvinyl polypyrrolidone (PVPP), polyvinyl pyrrolidone (PVP), hydroxypropyl methylcellulose phthalate (HPMCP), hypromellose acetate succinate (HPMCAS), ethylcellulose, croscamolluse sodium (CMS-Na), Starch 1500, carboxymethyl cellulose sodium (CMC-Na), methacrylic acid copolymer, methyl methacrylate copolymer, and a polymer comprising methacrylic acid-methyl methacrylate copolymer. In further embodiments, the polymer is EUDRAGIT® L100 or EUDRAGIT® L30D 55. EUDRAGIT® polymers are well-known in the pharmaceutical industry. They are marketed by Pharma Polymers, a division of Evonik Industries, Darmstadt, Germany. In some embodiments, the polymer is PVPP XL-10.

In some embodiments, the first polymer of the plurality of third pellets is present in between about 0.1% to about 50% by weight. In other embodiments, the first polymer of the plurality of third pellets is present in between about 1% to about 40% by weight, or between about 1% to about 30% by weight, or between about 1% to about 10% by weight, or between about 3% to about 8% by weight, or between about 5% to about 7% by weight. In some embodiments, the first polymer is present in about 5%. In some embodiments, the first polymer is present in about 6%. In some embodiments, the first polymer is present in about 7%.

In some embodiments, the core of the plurality of third pellets further comprises a second polymer. In some of these embodiments, the second polymer is a polymeric sugar. Polymeric sugars, or polysaccharides, include those sugars that are linked together through α-1,4 glycosidic bonds or β-1,4 glycosidic bonds. Examples of polymeric sugars include starch, glycogen, and cellulose. In some embodiments, the second polymer is cellulose. In some of these embodiments, the cellulose is derivatized, e.g., alkylated. In some embodiments, the cellulose is microcrystalline cellulose (PH101).

In some embodiments, the second polymer is present in between about 0.1% to about 50% by weight. In other embodiments, the second polymer is present in between about 1% to about 40% by weight, or between about 5% to about 30% by weight, or between about 5% to about 20% by weight, or between about 5% to about 15% by weight, or between about 8% to about 13% by weight. In some embodiments, the second polymer is present in about 8%. In some embodiments, the second polymer is present in about 9%. In some embodiments, the second polymer is present in about 11%.

In some embodiments, the core of the plurality of third pellets is made by passing 5-ASA, and the plurality of first polymers through micron screening sieves; whereupon they are weighed out into high shear granulator and mixed; a wetting agent is added to the mixture of 5-ASA and first polymers; the wet mixture is granulated; the granulated wet mass is then extruded using an extrusion hole; and the extruded material is spheronized and oven dried. In some embodiments the wetting agent used is water.

The sustained release formulations disclosed herein comprise two coatings. The sustained release coating is first coated over the core. The enteric coating is coated over the sustained-release-coated pellets. The enteric coating is dissolved when the pH of the medium reaches about 6.8, thereby exposing the sustained release coating. The sustained release coating then slowly dissolves to expose the core to the intestinal juices as the pellets move further down the GI tract.

In some embodiments, the enteric coating of the plurality of third pellets is substantially insoluble in media with pH<3. In other embodiments, the enteric coating of the plurality of first pellets is substantially insoluble in media with pH<4, or pH<5, or pH<6. In some embodiments, the enteric coating of the plurality of third pellets is substantially insoluble in media with pH<6.8.

In some embodiments, the first sustained release coating of the plurality of third pellets comprises a polymer substantially insoluble in gastric juice. In some of these embodiments, the polymer comprises ammonio methacrylate copolymer. In certain embodiments, the first sustained release coating comprises a polymer selected from the group consisting of EUDRAGIT® RL 100, EUDRAGIT® RL PO, EUDRAGIT® RL 12,5, EUDRAGIT® RL 30 D, EUDRAGIT® RS 100, EUDRAGIT® RS PO, EUDRAGIT® RS 12,5, and EUDRAGIT® RS 30 D. In certain embodiments, the first sustained release coating comprises a first polymer selected from the group consisting of EUDRAGIT® RL 100, EUDRAGIT® RL PO, EUDRAGIT® RL 12,5, and EUDRAGIT® RL 30 D, and a second polymer selected from the group consisting of EUDRAGIT® RS 100, EUDRAGIT® RS PO, EUDRAGIT® RS 12,5, and EUDRAGIT® RS 30 D.

In some embodiments, the first and second polymers are each independently present in between about 0.1% to about 50% by weight. In some of these embodiments, the first and second polymers are each independently present in between about 0.1% to about 40% by weight, or are each independently present in between about 0.1% to about 30% by weight, or are each independently present in between about 1% to about 10% by weight, or are each independently present in between about 1% to about 9% by weight, or are each independently present in between about 1% to about 3% by weight. In some embodiments, the first and second polymers are each independently present in about 1.1%. In some embodiments, the first and second polymers are each independently present in about 1.9%. In some embodiments, the first and second polymers are each independently present in about 2.5%.

In some embodiments, the first sustained release coating further comprises a pharmaceutically acceptable plasticizer. In some of these embodiments, the plasticizer is an alkyl citrate. In certain embodiments, the alkyl citrate is selected from the group consisting of triethyl citrate (TEC), acetyl triethyl citrate (ATEC), tributyl citrate (TBC), acetyl tributyl citrate (ATBC), trioctyl citrate (TOC), acetyl trioctyl citrate (ATOC), trihexyl citrate (THC), acetyl trihexyl citrate (ATHC), butyryl trihexyl citrate (BTHC, trihexyl o-butyryl citrate), and trimethyl citrate (TMC). In some embodiments, the plasticizer is triethyl citrate (TEC).

In some embodiments, the plasticizer is present in between about 0.1% to about 50% by weight. In some of these embodiments, the plasticizer is present in between about 0.1% to about 20% by weight, or between about 0.1% to about 10% by weight, or between about 0.1% to about 5% by weight, or between about 0.5% to about 3% by weight, or between about 0.5% to about 2% by weight. In some embodiments, the plasticizer is present in about 0.6%. In some embodiments, the plasticizer is present in about 1.0%. In some embodiments, the plasticizer is present in about 2.0%.

In some embodiments, the first sustained release coating further comprises an inert mineral. In some embodiments, the inert material is a nonionic water soluble surfactant and emulsifier like polysorbate (Tween 80). In some of these embodiments, the inert mineral is a mineral of magnesium. In certain embodiments, the mineral of magnesium is magnesium silicate. In some embodiments, the first sustained release coating further comprises talc.

In some embodiments, the inert mineral is present in between about 0.1% to about 50% by weight. In some of these embodiments, the inert mineral is present in between about 0.1% to about 40% by weight, or between about 0.5% to about 30% by weight, or between about 0.5% to about 10% by weight, or between about 1% to about 4% by weight, or between about 1.5% to about 3.5% by weight. In some embodiments, the inert mineral is present in about 1.5%. In some embodiments, the inert mineral is present in about 1.9%. In some embodiments, the inert mineral is present in about 2.4%. In some embodiments, the inert mineral is present in about 3.5%.

In some embodiments, the first sustained release coating of the plurality of third pellets is prepared by dispersing Triethyl citrate in a wetting agent, followed by adding talc to the triethyl citrate dispersion; the dispersion is homogenized; Eudragit RL 30 D and Eudragit RS 30 D are added slowly to the talc/triethyl citrate dispersion while stirring to maintain a uniformly dispersed sustained release matrix before coating it on the core of the plurality of third pellets as described below.

In some embodiments, the core of the third pellets is coated with the first sustained release coating in a fluid bed using bottom-spray technique with a spray nozzle.

In some embodiments, the third enteric coating of the plurality of third pellets comprises a polymer insoluble in gastric juice. In some of these embodiments, the polymer comprises methacrylic acid copolymer, methyl methacrylate copolymer, and a polymer comprising methacrylic acid-methyl methacrylate copolymer. In certain embodiments, the third enteric coating comprises EUDRAGIT S100.

In some embodiments, the polymer is present in between about 0.1% to about 50% by weight. In some of these embodiments, the polymer is present in between about 1% to about 40% by weight, or between about 1% to about 30% by weight, or between about 1% to about 10% by weight, or between about 3% to about 9% by weight, or between about 6% to about 8% by weight. In some embodiments, the polymer is present in about 6.8%. In some embodiments, the polymer is present in about 7.8%. In some embodiments, the polymer is present in about 8.8%.

In some embodiments, the third enteric coating further comprises a pharmaceutically acceptable plasticizer. In some of these embodiments, the plasticizer is an alkyl citrate. In certain embodiments, the alkyl citrate is selected from the group consisting of triethyl citrate (TEC), acetyl triethyl citrate (ATEC), tributyl citrate (TBC), acetyl tributyl citrate (ATBC), trioctyl citrate (TOC), acetyl trioctyl citrate (ATOC), trihexyl citrate (THC), acetyl trihexyl citrate (ATHC), butyryl trihexyl citrate (BTHC, trihexyl o-butyryl citrate), and trimethyl citrate (TMC). In some embodiments, the plasticizer is triethyl citrate (TEC).

In some embodiments, the plasticizer is present in between about 0.1% to about 50% by weight. In some of these embodiments, the plasticizer is present in between about 0.1% to about 20% by weight, or between about 0.1% to about 10% by weight, or between about 0.1% to about 1% by weight, or between about 0.3% to about 0.9% by weight, or between about 0.6% to about 0.8% by weight. In some embodiments, the plasticizer is present in about 0.6%. In some embodiments, the plasticizer is present in about 0.8%. In some embodiments, the plasticizer is present in about 0.9%.

In some embodiments, the third enteric coating further comprises an inert mineral. In some of these embodiments, the inert mineral is a mineral of magnesium. In certain embodiments, the mineral of magnesium is magnesium silicate. In some embodiments, the third enteric coating further comprises talc.

In some embodiments, the inert mineral is present in between about 0.1% to about 50% by weight. In some of these embodiments, the inert mineral is present in between about 0.1% to about 40% by weight, or between about 0.5% to about 30% by weight, or between about 0.5% to about 10% by weight, or between about 0.5% to about 2% by weight, or between about 0.8% to about 1.5% by weight. In some embodiments, the inert mineral is present in about 0.8%. In some embodiments, the inert mineral is present in about 1.5%. In some embodiments, the inert mineral is present in about 1.8%.

In some embodiments, the enteric matrix of the plurality of third pellets is prepared by dispersing Triethyl citrate in a wetting agent; talc is added to the triethyl citrate dispersion; the dispersion is homogenized; Eudragit S 100 is added slowly to the talc/triethyl citrate dispersion while stirring to maintain a uniformly dispersed enteric matrix before coating it on the first sustained release coating of the plurality of third pellets as described below.

In some embodiments, the first sustained release coating of the plurality of third pellets are enteric coated in a fluid bed using bottom-spray technique with a spray nozzle.

In some embodiments, the plurality of enteric coated first sustained release coat containing the core of the plurality of third pellets is cured in a fluid bed. In other embodiments, the pellets are cured in an oven; using SiO₂ to prevent pellet adhesion in the oven.

In another aspect, disclosed herein are a plurality of fourth pellets each comprising:
a) a core comprising N-acetylcysteine, or a pharmaceutically acceptable salt or ester thereof, and a first polymer;
b) a second sustained release coating substantially insoluble in gastric juice; and
c) a fourth enteric coating substantially insoluble in gastric juice.

In some embodiments, the N-acetylcysteine, or a pharmaceutically acceptable salt or ester thereof, in the plurality of fourth pellets is present in between about 10% to about 90% by weight. In some embodiments, the N-acetylcysteine, or a pharmaceutically acceptable salt or ester thereof, is present in between about 30% to about 90% by weight, or between about 50% to about 90% by weight, or between about 60% to about 90% by weight, or between about 60% to about 80% by weight, or between about 70% to about 80% by weight. In some embodiments, the N-acetylcysteine, or a pharmaceutically acceptable salt or ester thereof, is present in about 45%. In some embodiments, the N-acetylcysteine, or a pharmaceutically acceptable salt or ester thereof, is present in about 50%. In some embodiments, the N-acetylcysteine, or a pharmaceutically acceptable salt or ester thereof, is present in about 67%.

In some embodiments, the first polymer of the plurality of fourth pellets swells in aqueous media. In some of these embodiments, the first polymer of the plurality of second pellets is a cross-linked polymer. In certain embodiments, the first polymer of the plurality of second pellets is selected from the group consisting of polyvinyl polypyrrolidone (PVPP), polyvinyl pyrrolidone (PVP), hydroxypropyl methylcellulose phthalate (HPMCP), hypromellose acetate succinate (HPMCAS), ethylcellulose, croscamolluse sodium (CMS-Na), Starch 1500, methacrylic acid copolymer, methyl methacrylate copolymer, and a polymer comprising methacrylic acid-methyl methacrylate copolymer. In some embodiments, the polymer is EUDRAGIT® L100 or EUDRAGIT® L30D 55. In certain embodiments, the polymer is PVPP-XL10.

In some embodiments, the first polymer of the plurality of fourth pellets is present in between about 0.1% to about 50% by weight. In certain embodiments, the first polymer of the plurality of fourth pellets is present in between about 1% to about 40% by weight, or between about 1% to about 30% by weight, or between about 1% to about 10% by weight, or between about 3% to about 8% by weight, or between about 3% to about 6% by weight. In some embodiments, the first polymer is present in about 1.4%. In some embodiments, the first polymer is present in about 3.7%. In some embodiments, the first polymer is present in about 6.0%.

In some embodiments, the core of the plurality of fourth pellets further comprises a second polymer. In certain embodiments, the second polymer is a polymeric sugar, as defined above. In certain embodiments, the second polymer is cellulose, which can be microcrystalline cellulose.

In some embodiments, the second polymer is present in between about 0.1% to about 50% by weight. In certain embodiments, the second polymer is present in between about 1% to about 40% by weight, or between about 5% to about 30% by weight, or between about 5% to about 20% by weight, or between about 5% to about 15% by weight, or between about 8% to about 15% by weight. In some embodiments, the second polymer is present in about 5.2%. In some embodiments, the second polymer is present in about 9.7%. In some embodiments, the second polymer is present in about 12.2%.

In some embodiments, the core of the plurality of fourth pellets can comprise a third polymer. In certain embodiments, the third polymer is a polymeric sugar, as defined above. In certain embodiments, the third polymer is cellulose, which can be ethylene cellulose (EC100 cps) or HPMC (K4M). In certain embodiments, the third polymer is polyvinyl pyrilodone, which can be K-29/32, S-630/32 or S-630.

In some embodiments, the third polymer is present in between about 0.1% to about 50% by weight. In certain embodiments, the second polymer is present in between about 1% to about 40% by weight, or between about 5% to about 30% by weight, or between about 5% to about 20% by weight, or between about 5% to about 15% by weight, or between about 8% to about 15% by weight.

In some embodiments, the core of the plurality of fourth pellets further comprises an inert matrix forming material. In some embodiments, the inert matrix forming material is selected from a group of amphiphilic materials with high melting point. In certain embodiments, the inert matrix forming material is glycerol behenate (Compritol 888ATO).

In some embodiments, the inert matrix forming material is present in between about 0.1% to about 50% by weight. In certain embodiments, the second polymer is present in between about 1% to about 40% by weight, or between about 5% to about 30% by weight, or between about 5% to about 20% by weight, or between about 5% to about 15% by weight, or between about 8% to about 15% by weight. In some embodiments, the inert matrix forming material is present in about 8.1%. In some embodiments, the inert matrix forming material is present in about 9.7%. In some embodiments, the inert matrix forming material is present in about 13.1%. In some embodiments, the inert matrix forming material is present in about 14.2%.

In some embodiments, the core of the plurality of fourth pellets further comprises an antioxidant. In certain embodiments, the antioxidant is selected from the group consisting of ascorbic acid, propyl gallate, tocopherols, tertiary butylhydroquinone, butylated hydroxyanisole and butylated hydroxytoluene. In some of these embodiments, the antioxidant is ascorbic acid.

In some embodiments, the antioxidant is present in between about 0.1% to about 50% by weight. In some of these embodiments, the antioxidant is present in between about 0.1% to about 20% by weight, or between about 0.1% to about 10% by weight, or between about 0.1% to about 1% by weight, or between about 0.1% to about 0.6% by weight, or between about 0.1% to about 0.4% by weight. In some embodiments, the antioxidant is present in about 0.2%. In some embodiments, the antioxidant is present in about 0.4%. In some embodiments, the antioxidant is present in about 0.5%.

In some embodiments, the core of the plurality of fourth pellets is made by passing NAC, the first polymer, the second polymer, the inert matrix material and the antioxidant through micron screening sieves; weighed out into high shear granulator and mixed; added a wetting agent to the mixture of NAC, the first polymer, the second polymer, the inert matrix material and the antioxidant; the wet mixture granulated; the granulated wet mass then extruded using extrusion hole; the extruded material spheronized, and oven dried. In some embodiments the wetting agent used is isporopyl alcohol. In certain embodiments the wetting agent used is water.

In some embodiments, the second sustained release coating of the plurality of fourth pellets comprises a polymer insoluble in gastric juice. In some of these embodiments, the polymer comprises ammonio methacrylate copolymer. In certain embodiments, the second sustained release coating comprises a first polymer selected from the group consisting of EUDRAGIT® RL 100, EUDRAGIT® RL PO, EUDRAGIT® RL 12,5, and EUDRAGIT® RL 30 D, EUDRAGIT® RL 100, EUDRAGIT® L 100-55, EUDRAGIT® L 30 D-55, EUDRAGIT® L100, EUDRAGIT® S100, EUDRAGIT® FS 30D, EUDRAGIT® RL PO, EUDRAGIT® RL 12,5, EUDRAGIT® RL 30 D, EUDRAGIT® RS 100, EUDRAGIT® RS PO, EUDRAGIT® RS 12,5, and EUDRAGIT® RS 30 D. In certain embodiments, first polymer is Eudragit L100.

In some embodiments, the second sustained release coating further comprises a second polymer polymer selected from the group consisting of, polymeric sugar molecules. In certain embodiments, the second polymer of the second sustained release coating of the plurality of fourth pellets is ethyl cellulose (EC 20 cps).

In some embodiments, the first and second polymers of the second sustained release coating of the plurality of fourth pellets are each independently present in between about 0.1% to about 50% by weight. In some of these embodiments, the first and second polymers are each independently present in between about 0.1% to about 40% by weight, or are each independently present in between about 0.1% to about 30% by weight, or are each independently present in between about 1% to about 10% by weight, or are each independently present in between about 1% to about 9% by weight, or are each independently present in between about 2% to about 5% by weight. In some embodiments, the first and second polymers are each independently present in about 2%. In some embodiments, the first and second polymers are each independently present in about 3%. In some embodiments, the first and second polymers are each independently present in about 4%.

In some embodiments, the second sustained release coating further comprises a pharmaceutically acceptable plasticizer. In some of these embodiments, the plasticizer is an alkyl citrate. In certain embodiments, the alkyl citrate is selected from the group consisting of triethyl citrate (TEC), acetyl triethyl citrate (ATEC), tributyl citrate (TBC), acetyl tributyl citrate (ATBC), trioctyl citrate (TOC), acetyl trioctyl citrate (ATOC), trihexyl citrate (THC), acetyl trihexyl citrate (ATHC), butyryl trihexyl citrate (BTHC, trihexyl o-butyryl citrate), and trimethyl citrate (TMC). In some embodiments, the plasticizer is triethyl citrate (TEC).

In some embodiments, the plasticizer is present in between about 0.1% to about 50% by weight. In some of these embodiments, the plasticizer is present in between about 0.1% to about 20% by weight, or between about 0.1% to about 10% by weight, or between about 0.1% to about 5% by weight, or between about 0.5% to about 3% by weight, or between about 0.5% to about 2% by weight. In some embodiments, the plasticizer is present in about 0.9%. In some embodiments, the plasticizer is present in about 1.4%. In some embodiments, the plasticizer is present in about 2.6%.

In some embodiments, the second sustained release coating further comprises an inert mineral. In some of these embodiments, the inert mineral is a mineral of magnesium. In certain embodiments, the mineral of magnesium is magnesium silicate. In some embodiments, the second sustained release coating further comprises talc.

In some embodiments, the inert mineral is present in between about 0.1% to about 50% by weight. In some of these embodiments, the inert mineral is present in between about 0.1% to about 40% by weight, or between about 0.5% to about 30% by weight, or between about 0.5% to about 10% by weight, or between about 1% to about 4% by weight, or between about 2% to about 4% by weight. In some embodiments, the inert mineral is present in between about 2.9%. In some embodiments, the inert mineral is present in between about 3.5%. In some embodiments, the inert mineral is present in between about 4.0%.

In some embodiments, the second sustained release matrix of the plurality of fourth pellets is prepared by dispersing triethyl citrate in a wetting agent; talc is added to the triethyl citrate dispersion; the dispersion is homogenized; EC (20 cps) and Eudragit L100 are added slowly to the talc/triethyl citrate dispersion while stirring to maintain a uniformly dispersed sustained release matrix before coating it on the core of the plurality of third pellets as described below.

In some embodiments, the core of the fourth pellets are coated with the second sustained release coating in a fluid bed using bottom-spray technique with a spray nozzle.

In some embodiments, the fourth enteric coating of the plurality of fourth pellets is substantially insoluble in media with pH<3. In other embodiments, the enteric coating of the plurality of second pellets is substantially insoluble in media with pH<4, or pH<5, or pH<6. In some embodiments, the enteric coating of the plurality of second pellets is substantially insoluble in media with pH<6.5.

In some embodiments, the fourth enteric coating of the plurality of fourth pellets comprises a polymer insoluble in gastric juice. In some of these embodiments, the polymer comprises methacrylic acid copolymer, methyl methacrylate copolymer, and a polymer comprising methacrylic acid-methyl methacrylate copolymer. In certain embodiments, the enteric coating comprises EUDRAGIT® S100.

In some embodiments, the polymer is present in between about 0.1% to about 50% by weight. In some of these embodiments, the polymer is present in between about 1% to about 40% by weight, or between about 1% to about 30% by weight, or between about 1% to about 15% by weight, or between about 3% to about 12% by weight, or between about 6% to about 12% by weight. In some embodiments, the polymer is present in about 6.1%. In some embodiments, the polymer is present in about 9.4%. In some embodiments, the polymer is present in about 13.8%.

In some embodiments, the fourth enteric coating further comprises a pharmaceutically acceptable plasticizer. In some of these embodiments, the plasticizer is an alkyl citrate. In certain embodiments, the alkyl citrate is selected from the group consisting of triethyl citrate (TEC), acetyl triethyl citrate (ATEC), tributyl citrate (TBC), acetyl tributyl citrate (ATBC), trioctyl citrate (TOC), acetyl trioctyl citrate (ATOC), trihexyl citrate (THC), acetyl trihexyl citrate (ATHC), butyryl trihexyl citrate (BTHC, trihexyl o-butyryl citrate), and trimethyl citrate (TMC). In some embodiments, the plasticizer is triethyl citrate (TEC).

In some embodiments, the plasticizer is present in between about 0.1% to about 50% by weight. In some of these embodiments, the plasticizer is present in between about 0.1% to about 20% by weight, or between about 0.1% to about 10% by weight, or between about 0.1% to about 1% by weight, or between about 0.3% to about 1% by weight, or between about 0.6% to about 1% by weight. In some embodiments, the plasticizer is present in about 0.4%. In some embodiments, the plasticizer is present in about 1.5%. In some embodiments, the plasticizer is present in about 2.6%.

In some embodiments, the fourth enteric coating further comprises an inert mineral. In some of these embodiments, the inert mineral is a mineral of magnesium. In certain embodiments, the mineral of magnesium is magnesium silicate. In some embodiments, the fourth enteric coating further comprises talc.

In some embodiments, the inert mineral is present in between about 0.1% to about 50% by weight. In some of these embodiments, or between about 0.1% to about 40% by weight, or between about 0.5% to about 30% by weight, or between about 0.5% to about 10% by weight, or between about 0.5% to about 2% by weight, or between about 1% to about 2% by weight. In some embodiments, the inert mineral is present in about 1.9%. In some embodiments, the inert mineral is present in about 2.3%. In some embodiments, the inert mineral is present in about 2.9%.

In some embodiments, the enteric coating of the plurality of fourth pellets is prepared by dispersing Triethyl citrate in a wetting agent; talc is added to the triethyl citrate dispersion; the dispersion is homogenized; Eudragit S 100 is added slowly to the talc/triethyl citrate dispersion while stirring to maintain a uniformly dispersed enteric matrix before coating it on the second sustained release coating of the plurality of fourth pellets as described below.

In some embodiments, the second sustained release coating of the plurality of fourth pellets are enteric coated in a fluid bed using bottom-spray technique with a spray nozzle.

In some embodiments, the plurality of enteric coated second sustained release coat containing the core of the plurality of fourth pellets is cured in a fluid bed. In other embodiments, the pellets are cured in an oven; using SiO₂ to prevent pellet adhesion in the oven.

In certain of the aspects disclosed above, the particle size of all APIs (e.g., 5-ASA and NAC) and excipients is controlled. In some embodiments, NAC particle size is limited to a distribution of sizes in the range 90-500 microns, with the majority of particles in the size range 125-355 microns, and the peak of the distribution being between 120-255 microns. In some embodiments, particle sizes of less than 180 micron are used and the larger particles are screened out, whereas in other embodiments, all the particles within the above distribution range are used.

In another aspect, disclosed herein are pharmaceutical compositions comprising one of the following combinations of the above pellets: a) a plurality of the first pellets and a plurality of the second pellets; b) a plurality of the first pellets and a plurality of the third pellets; c) a plurality of the first pellets and a plurality of the fourth pellets; d) a plurality of the second pellets and a plurality of the third pellets; e) a plurality of the second pellets and a plurality of the fourth pellets; f) a plurality of the third pellets and a plurality of the fourth pellets; g) a plurality of the first pellets, a plurality of the second pellets, and a plurality of the third pellets; h) a plurality of the first pellets, a plurality of the second pellets, and a plurality of the fourth pellets; i) a plurality of the first pellets, a plurality of the third pellets, and a plurality of the fourth pellets; j) a plurality of the second pellets, a plurality of the third pellets, and a plurality of the fourth pellets; and k) a plurality of the first pellets, a plurality of the second pellets, a plurality of the third pellets, and a plurality of the fourth pellets.

In another aspect, disclosed herein are capsules comprising a plurality of the pellets, as defined above. In another aspect, disclosed herein are capsules comprising one of the combinations of pellets, as disclosed above.

In some embodiments, the capsule is soluble in an aqueous solution having a pH less than 5, but is substantially insoluble in an aqueous solution having a pH greater than 7.

In another aspect, disclosed herein are dose sipping straws comprising a plurality of the pellets, as defined above. In another aspect, disclosed herein are dose sipping straws comprising one of the combinations of pellets, as disclosed above. In some embodiments, the pellets are filled into a straw and a patient then drinks liquid through the straw, and through the process of drinking, the liquid pulled through the straw brings the pellets into the mouth along with the liquid.

In another aspect, disclosed herein are dry sachets comprising a plurality of the pellets, as defined above. In another aspect, disclosed herein are dry sachets comprising one of the combinations of pellets, as disclosed above. In some embodiments, the pellets will be sprinkled onto food or mixed into a drink from dry sachet, and taken orally. For the dosage to be effective, the disclosed pellets are filled into a sachet pouch, along with additional excipients needed to form a readily dispersible suspension. When the pouch is opened and the contents are poured over food or into a drink, the pellets and additional excipients are mixed with the food or the drink, and form a palatable dispersion that is ingested by the subject. Excipients, such as salivants and glidants, are added for the contents to be easily swallowed with a minimum of chewing so that the enteric and sustained release coatings are not broken in the mouth.

In another aspect, disclosed herein are ready-to-use sachets comprising a plurality of the pellets, as defined above. In another aspect, disclosed herein are ready-to-use sachets comprising one of the combinations of pellets, as disclosed above. In some embodiments, the pellets are premixed with an edible, high viscosity food substance (for example, yogurt, or energy gel), and the entire contents of the package is taken orally. Excipients, such as salivants and glidants, are added for the contents to be easily swallowed with a minimum of chewing so that the enteric and sustained release coatings are not broken in the mouth.

In another aspect, disclosed herein are tablets comprising a plurality of the pellets, as defined above. In another aspect, disclosed herein are tablets comprising one of the combinations of pellets, as disclosed above. In some embodiments, the plurality of the pellets are pressed together using binders, glidants, or other excipients. In certain embodiments, the tablet comprises a coating, for example an enteric coating as described above. The subject takes the tablet and the coating dissolves in the gut, whereupon the pellets are released.

In another embodiment, the pellets are compressed into a large dissolvable tablet. The tablet is then placed in a potable liquid, such as water or some other drink. One or more excipients are used to allow the tablet to dissolve quickly and form a high viscosity suspension when stirred. In some embodiments, the tablet material includes salivants or glidants so that the contents are easily swallowed with a minimum of chewing.

In another aspect, disclosed herein are mini-tablets comprising a plurality of the pellets, as defined above. In another aspect, disclosed herein are mini-tablets comprising one of the combinations of pellets, as disclosed above. The mini-tablets are produced in the same manner as described for the tablets, with the exception that while a tablet contains a single administrable dose of the 5-ASA and/or NAC, a mini-tablet contains less than a single administrable dose. The mini-tablets are then pressed together into a tablet, or contained in a capsule, where the combination of the several mini-tablets in a single tablet or in a single capsule forms a single administrable dose.

In another aspect, disclosed herein are suspensions comprising a plurality of the pellets, as defined above. In another aspect, disclosed herein are suspensions comprising one of the combinations of pellets, as disclosed above. In some embodiments, the suspensions comprise ingredients such as glycerin, microcrystalline cellulose, carboxymethyl cellulose sodium, sorbitol solution, xanthan gum, and the like, and various colorings and flavorings to make the suspension palatable for pediatric use.

In some embodiments, the pharmaceutical formulation forms disclosed above comprise a plurality of pellets selected from the group consisting of the following: a) a plurality of the first pellets and a plurality of the second pellets;) a plurality of the first pellets and a plurality of the third pellets; c) a plurality of the first pellets and a plurality of the fourth pellets; d) a plurality of the second pellets a plurality of the third pellets; e) a plurality of the second pellets and a plurality of the fourth pellets; f) a plurality of the third pellets and a plurality of the fourth pellets; g) a plurality of the first pellets, a plurality of the second pellets, and a plurality of the third pellets; h) a plurality of the first pellets, a plurality of the second pellets, and a plurality of the fourth pellets; i) a plurality of the first pellets, a plurality of the third pellets, and a plurality of the fourth pellets; j) a plurality of the second pellets, a plurality of the third pellets, and a plurality of the fourth pellets; and k) a plurality of the first pellets, a plurality of the second pellets, a plurality of the third pellets, and a plurality of the fourth pellets; l) a plurality of the first pellets; m) a plurality of the second pellets; n) a plurality of the third pellets; and o) a plurality of the fourth pellets.

### EXAMPLES

### Example 1

### 5-ASA Enteric Immediate Release Pellet Manufacturing Process

Formation of 5-ASA Core Pellets: 5-ASA, PH101, and PVPP-XL10 were passed through 180 micron screening sieves, and the required amounts of each were weighed out into a high shear granulator and mixed at 500 rpm for 3 minutes. Water was added to the mixture at a rate of 100 mL/min and the wet mixture was granulated for 8 minutes at 500 rpm. The wet mass was then extruded with an extrusion speed of 30 rpm using extrusion hole diameters of 1.0 mm. The extruded material was spheronized at a speed of 800 rpm for 10 minutes, and oven dried at a temperature of 50° C. Beads were primarily in the 800-900 micron size range.

Several batches with the following amounts of ingredients were prepared:

1) 5-ASA: 400 g, PH101: 62.5 g, PVPP-XL10: 37.5 g, water: 300 g.

2) 5-ASA: 150 g, PH 101: 44 g, CMS-Na: 6 g, water: 100 g.

3) 5-ASA: 160 g, PH 101: 29.5 g, Starch 1500: 10 g, CMC-Na: 0.5 g, water: 75 g.

4) 5-ASA: 160 g; PH101: 25-30 g; PVPP-XL 10: 10-15 g; Water: 105-120 g.

Preparation of 5-ASA Enteric Coating Solution: Triethyl citrate was dispersed in 95% ethanol, then talc was added to the triethyl citrate dispersion. The dispersion was homogenized for 5-15 min at 10,000 rpm. Eudragit S 100 was added slowly to the talc/triethyl citrate dispersion while stirring at 300-600 rpm. The coating dispersion was stirred continuously until the coating process was finished.

In some batches, the amount of coating as a percent weight of the coated pellet was as follows: 6%, 8%, 10%, 12%, or 15%, without a barrier coat, or 6%, 8%, 10%, 12%, 14%, or 15%, with a barrier coat.

Several batches with the following amounts of ingredients were prepared:

1) Triethyl citrate: 0.8 g, talc: 1.6 g, Eudragit S 100: 8.0 g, 95% ethanol: 89.6 g.

2) Eudragit S 100: 15.0 g, Eudragit L 100: 15.0 g, TEC: 3.0 g, talc: 6.0 g.

3) Eudragit FS: 50.0 g, talc: 25.0 g.

4) Eudragit FS: 70.0 g, talc: 35.0 g.

5-ASA Barrier Coating: In some cases, a barrier coating was applied to the core pellets before the enteric coating was added. The barrier was a solution of HPMC:PEG 400 (1:1 weight ratio). The barrier coating was applied in a fluid bed using a process similar to the enteric coating procedure. The barrier was applied such that the percentage by weight of the barrier coat was 2.0%.

5-ASA Enteric Coating Procedure: Core pellets or barrier coated pellets were enteric coated in a fluid bed using bottom-spray technique with the following process parameters: material charge: 150-200 g; spray nozzle diameter: 0.5 mm; air pressure: 0.5-0.7 bar; atomizing pressure: 1.0 bar; flow rate: 4-6 g/min; inlet temperature: 30-40° C.; product temperature: 23-25° C.

Product Curing: 5-ASA EIR coated pellets were cured for 16 hours at 40° C., either in the fluid bed or in an oven. When an oven was used, 1% (w/w) SiO₂ was added to prevent pellet adhesion.

### Example 2

### 5-ASA Enteric Sustained Release Pellet Manufacturing Process

Formation of 5-ASA Core Pellets: 5-ASA, PH101, and PVPP-XL10 were passed through 180 micron screening sieves, and the required amounts of each were weighed out into a high shear granulator and mixed at 500 rpm for 3 minutes. Water was added to the mixture at a rate of 100 mL/min and the wet mixture was granulated for 8 minutes at 500 rpm. The wet mass was extruded with an extrusion speed of 30 rpm using extrusion hole diameters of 1.0 mm. The extruded material was spheronized at a speed of 800 rpm for 10 minutes, and oven dried at a temperature of 50° C. Beads were primarily in the 800-900 micron size range.

A batch with the following amounts of ingredients was prepared: 5-ASA: 400 g, PH101: 62.5 g, PVPP-XL10: 37.5 g, water: 300 g.

Preparation of 5-ASA Sustained Release Coating Solution: Triethyl citrate was dispersed in water, then talc was added to the triethyl citrate dispersion. The dispersion was homogenized for 5-15 min at 10,000 rpm. Eudragit RL 30 D and Eudragit RS 30 D were added slowly to the talc/triethyl citrate dispersion while stirring at 300-600 rpm. The coating dispersion was stirred continuously until the coating process was finished.

In some batches, the amount of coating as a percent weight of the coated pellet was as follows: 6%, 8%, 10% or 14%, without a barrier coat.

Several batches with the following amounts of ingredients were prepared:

1) Triethyl citrate: 6.0 g, talc: 15.0 g, Eudragit RL 30: 15.0 g, Eudragit RS 30: 15.0 g, water: 134.0 g.

2) Eudragit RL: 50.0 g, Eudragit RS: 5.0 g, TEC: 10.0 g, and talc: 25.0 g.

3) Eudragit RL: 15.0 g, Eudragit RS: 15.0 g, TEC: 6.0 g, and talc: 15.0 g.

4) Eudragit RL: 15.0 g, Eudragit RS: 35.0 g, TEC: 10.0 g, Tween 80: 1.0 g, and GMS: 5.0 g.

Preparation of 5-ASA Enteric Coating Solution: Triethyl citrate was dispersed in 95% ethanol, then talc was added to the triethyl citrate dispersion. The dispersion was homogenized for 5-15 min at 10,000 rpm. Eudragit S 100 was added slowly to the talc/triethyl citrate dispersion while stirring at 300-600 rpm. The coating dispersion was stirred continuously until the coating process was finished.

In a batch size of 100 g, the amounts of ingredients added were: Triethyl citrate: 0.8 g, talc: 1.6 g, Eudragit S 100: 8.0 g, 95% ethanol: 89.6 g.

5-ASA Sustained Release Coating Procedure: Core pellets were coated in a fluid bed using bottom-spray technique with the following process parameters: material charge: 150-200 g; spray nozzle diameter: 0.5 mm; air pressure: 0.5-0.7 bar; atomizing pressure: 1.0 bar; flow rate: 3-6 g/min; inlet temperature: 40-50° C.; product temperature: 25-28° C.

5-ASA Enteric Coating Procedure: Sustained release pellets were enteric coated in a fluid bed using bottom-spray technique with the following process parameters: material charge: 150-200 g; spray nozzle diameter: 0.5 mm; air pressure: 0.5-0.7 bar; atomizing pressure: 1.0 bar; flow rate: 4-6 g/min; inlet temperature: 30-40° C.; product temperature: 23-25° C.

Product Curing: 5-ASA ESR coated pellets were cured for 16 hours at 40° C., either in the fluid bed or in an oven. When an oven was used, 1% (w/w) SiO₂ was added to prevent pellet adhesion.

### Example 3

### NAC Enteric Immediate Release Pellet Manufacturing Process

Formation of NAC Core Pellets: NAC, PH101, PVPP-XL10, ascorbic acid, and Compritol 888 ATO were passed through 180 micron screening sieves, and the required amounts of each were weighed out into a high shear granulator and mixed at 500 rpm for 3 minutes. Water was added to the mixture at a rate of 100 mL/min and the wet mixture was granulated for 8 minutes at 500 rpm. The wet mass was extruded with an extrusion speed of 30 rpm using extrusion hole diameters of 1.2 mm. The extruded material was spheronized at a speed of 600 rpm for 5 minutes, and oven dried at a temperature of 40° C. Beads were primarily in the 800-1000 micron size range.

Several batches with the following amounts of ingredients were prepared:

1) NAC, PH101, PVPP-XL10, Compritrol 888 ATO, ascorbic acid, and water, at 400 g, 41.7 g, 11.4 g, 114.3 g, 4.0 g, 174.3 g, respectively.

2) NAC, PH 101, Starch 1500, and water, at 160 g, 30-32 g, 8-10 g, and 50-55 mL, respectively.

3) NAC, PH 101, Starch 1500, and 5% ethanol, at 160 g, 30 g, 10 g, and 50 mL, respectively.

4) NAC, PH 101, Starch 1500, and 10% ethanol, at 160 g, 30 g, 10 g, and 55 mL, respectively.

5) NAC, PH 101, Starch 1500, and 30% ethanol, at 160 g, 30 g, 10 g, and 55 mL, respectively.

6) NAC, PH 101, PVPP-XL 10, and water, at 160 g, 30 g, 10 g, and 60 mL, respectively.

7) NAC, PH 101, PVPP-XL 10, and water, at 160 g, 30 g, 10 g, and 73 mL, respectively.

8) NAC, PH 101, PVPP-XL 10, and water, at 160 g, 30 g, 10 g, and 80 mL, respectively.

9) NAC, ascorbic acid, PH 101, PVPP-XL 10, and water, at 80.0 g, 0.8 g, 14.2 g, 5.0 g, and 36.5 mL, respectively.

10) NAC, PH 101, PVPP-XL 10, and 30% isopropyl alcohol, at 80.0 g, 15.0 g, 5.0 g, and 44.0 mL, respectively.

11) NAC, PH 101, PVPP-XL 10, and 5% K-29/32 (binding agent) in water, at 80.0 g, 13.2 g, 5.0 g, and 36.0 mL, respectively.

12) NAC, PH 101, PVPP-XL 10, and 5% S-630/32 (binding agent) in water, at 80.0 g, 13.2 g, 5.0 g, and 36.5 mL, respectively.

13) NAC, PH 101, PVPP-XL 10, and 20% K-29/32 (binding agent) in water, at 80.0 g, 13.2 g, 5.0 g, and 26.0 mL, respectively.

14) NAC, PH 101, PVPP-XL 10, and 20% S-630/32 (binding agent) in water, at 80.0 g, 13.2 g, 5.0 g, and 26.0 mL, respectively.

15) NAC, ascorbic acid, PH 101, PVPP-XL 10, HPMC (K4M) and 30% isopropyl alcohol, at 140.0 g, 1.4 g, 8.6 g, 10.0 g, 40.0 g, and about 62 mL, respectively.

16) NAC, ascorbic acid, PH 101, PVPP-XL 10, EC (100 cps) and water, at 140.0 g, 1.4 g, 8.6 g, 10.0 g, 40.0 g, and about 62 mL, respectively.

17) NAC, ascorbic acid, PH 101, PVPP-XL 10, Compritol 888 ATO and water, at 140.0 g, 1.4 g, 8.6 g, 4.0 or 10.0 g, 40.0 g, and 62-63 mL, respectively.

18) NAC, ascorbic acid, PH 101, Compritol 888 ATO and water, at 140.0 g, 1.4 g, 18.6 g, 40.0 g, and 63 mL, respectively.

19) NAC, ascorbic acid, PH 101, PVPP XL-10, and water, at 140.0 g, 1.4 g, 18.6 g, 40.0 g, and 63 mL, respectively.

20) NAC, ascorbic acid, PH 101, PVPP-XL 10, and 30% isopropyl alcohol, at 400.0 g, 4.0 g, 71.0 g, 25.0 g, and 220.0 mL, respectively.

21) NAC, ascorbic acid, PH 101, PVPP-XL 10, and 5% S-630 in water, at 400.0 g, 4.0 g, 62.3 g, 25.0 g, and 175.0 mL, respectively.

22) NAC, ascorbic acid, PH 101, PVPP-XL 10, and 5% S-630 in 30% isopropyl alcohol, at 400.0 g, 4.0 g, 62.0 g, 25.0 g, and 175.0 mL, respectively.

NAC Barrier Coating: In some cases, a barrier coating was applied to the core pellets before the enteric coating was added. The barrier was either a solution of HPMC (E6):PEG 400 (10:1 weight ratio), or HPCM (E6):magnesium stearate (1:1 weight ratio). The barrier coating was applied in a fluid bed using a process similar to the enteric coating procedure. The barrier was applied such that the percentage by weight of the barrier coat was 2.0%.

Preparation of NAC Enteric Coating Solution: Triethyl citrate was dispersed in 95% ethanol, then talc was added to the triethyl citrate dispersion. The dispersion was homogenized for 5-15 min at 10,000 rpm. Eudragit S 100 was added slowly to the talc/triethyl citrate dispersion while stirring at 300-600 rpm. The coating dispersion was stirred continuously until the coating process was finished.

In a batch size of 100 g, the amounts of ingredients added were: Triethyl citrate: 0.8 g, talc: 1.6 g, Eudragit S 100: 8.0 g, 95% ethanol: 89.6 g.

NAC Enteric Coating Procedure: Core pellets were enteric coated in a fluid bed using bottom-spray technique with the following process parameters: material charge: 150-200 g; spray nozzle diameter: 0.5 mm; air pressure: 0.5-0.7 bar; atomizing pressure: 1.0 bar; flow rate: 4-6 g/min; inlet temperature: 30-40° C.; product temperature: 23-25° C.

Product Curing: NAC EIR coated pellets were cured for 16 hours at 50° C., either in the fluid bed or in an oven. When an oven was used, 1% (w/w) SiO₂ was added to prevent pellet adhesion.

### Example 4

### NAC Enteric Sustained Release Pellet Manufacturing Process

Formation of NAC Core Pellets: NAC, PH101, PVPP-XL10, ascorbic acid, and Compritol 888 ATO were passed through 180 micron screening sieves, and the required amounts of each were weighed out into a high shear granulator and mixed at 500 rpm for 3 minutes. Water was added to the mixture at a rate of 100 mL/min and the wet mixture was granulated for 8 minutes at 500 rpm. The wet mass was extruded with an extrusion speed of 30 rpm using extrusion hole diameters of 1.2 mm. The extruded material was spheronized at a speed of 600 rpm for 5 minutes, and oven dried at a temperature of 40° C. Beads were primarily in the 800-1000 micron size range.

In a batch size of 746 g, the amounts of ingredients added were: NAC: 400 g, PH101: 41.7 g, PVPP-XL10: 11.4 g, Compritrol 888 ATO: 114.3 g, ascorbic acid: 4.0 g, water: 174.3 g.

Preparation of NAC Sustained Release Coating Solution: Triethyl citrate was dispersed in 95% ethanol, then talc was added to the triethyl citrate dispersion. The dispersion was homogenized for 5-15 min at 10,000 rpm. EC (20 cps) and Eudragit L 100 were added slowly to the talc/triethyl citrate dispersion while stirring at 300-600 rpm. The coating dispersion was stirred continuously until the coating process was finished.

Several batches with the following amounts of ingredients were prepared:

1) Triethyl citrate, talc, EC (20 cps), Eudragit L 100, and 95% ethanol, at 0.9 g, 2.3 g, 11.0 g, 9.0 g, and 208.4 g, respectively.

Eudragit RS 30 D, triethyl citrate, talc, at 142.9 g, 8.6 g, and 71.4 g, 25% polymer added by weight.

Surelease (EC) at 15% of polymer added by weight.

Surelease (EC):PEG 6000, 6.5:3.5 weight ratio, at 8% polymer added by weight.

Surelease (EC):HPMC (E6), 6.5:3.5 weight ratio, at 8% polymer added by weight.

EC (20 cps):Eudragit RS 100, 3:7 weight ratio, at 15% polymer added by weight.

EC (20 cps):Eudragit RS 100, 7:3 weight ratio, at 9% polymer added by weight.

EC (20 cps):Eudragit S 100, 6.5:3.5 weight ratio, at 8% polymer added by weight.

EC (20 cps): Eudragit RS 100:Eudragit S 100, 6:3:3 weight ratio, at 8% polymer added by weight.

EC (20 cps):Eudragit L 100, 5:5 weight ratio, at 8% polymer added by weight.

EC (20 cps):Eudragit L 100, 5.5:4.5 weight ratio, at 8% polymer added by weight.

Preparation of NAC Enteric Coating Solution: Triethyl citrate was dispersed in 95% ethanol, then talc was added to the triethyl citract dispersion. The dispersion was homogenized for 5-15 min at 10,000 rpm. Eudragit S 100 was added slowly to the talc/triethyl citrate dispersion while stirring at 300-600 rpm. The coating dispersion was stirred continuously until the coating process was finished.

In a batch size of 100 g, the amounts of ingredients added were: Triethyl citrate: 0.8 g, talc: 1.6 g, Eudragit S 100: 8.0 g, 95% ethanol: 89.6 g.

NAC Sustained Release Coating Procedure: Core pellets were coated in a fluid bed using bottom-spray technique with the following process parameters: material charge: 150-200 g; spray nozzle diameter: 0.5 mm; air pressure: 0.5-0.7 bar; atomizing pressure: 1.0 bar; flow rate: 3-6 g/min; inlet temperature: 28-36° C.; product temperature: 23-28° C.

NAC Enteric Coating Procedure: Sustained release pellets were enteric coated in a fluid bed using bottom-spray technique with the following process parameters: material charge: 150-200 g; spray nozzle diameter: 0.5 mm; air pressure: 0.5-0.7 bar; atomizing pressure: 1.0 bar; flow rate: 4-6 g/min; inlet temperature: 30-40° C.; product temperature: 23-25° C.

Product Curing: NAC ESR coated pellets were cured for 16 hours at 50° C., either in the fluid bed or in an oven. When an oven was used, 1% (w/w) SiO₂ was added to prevent pellet adhesion.

What is claimed is:

1. A plurality of pellets selected from:
a plurality of first pellets each comprising:
a core comprising 5-aminosalicylic acid, or a pharmaceutically acceptable salt or ester thereof, and a first polymer; and
a first enteric coating; or
a plurality of third pellets each comprising:
a core comprising 5-aminosalicylic acid, or a pharmaceutically acceptable salt or ester thereof, and a first polymer;
a first sustained release coating; and
a third enteric coating.

2. A plurality of pellets selected from:
a plurality of second pellets each comprising:
a core comprising N-acetylcysteine, or a pharmaceutically acceptable salt or ester thereof, and a first polymer; and
a second enteric coating; or
a plurality of fourth pellets each comprising:
a core comprising N-acetylcysteine, or a pharmaceutically acceptable salt or ester thereof, and a first polymer;
a second sustained release coating; and
a fourth enteric coating.

3. The pellets of claim 1, wherein the plurality of first or third pellets comprises 5-aminosalicylic acid, or a pharmaceutically acceptable salt or ester thereof, in between about 30% to about 90% by weight.

4. The pellets of claim 1, wherein the first polymer is selected from the group consisting of polyvinyl polypyrrolidone (PVPP), polyvinyl pyrrolidone (PVP), hydroxypropyl methylcellulose phthalate (HPMCP), hypromellose acetate succinate (HPMCAS), ethylcellulose, croscamolluse sodium (CMS-Na), Starch 1500, carboxymethyl cellulose sodium (CMC-Na), methacrylic acid copolymer, methyl methacrylate copolymer, a polymer comprising methacrylic acid-methyl methacrylate copolymer, EUDRAGIT® L100 and EUDRAGIT® L30D 55.

5. The pellets of claim 1, wherein the core of the plurality of first or third pellets further comprises a second polymer selected from the group consisting of cellulose, alkylated cellulose, and microcrystalline cellulose.

6. The pellets of claim 1, wherein the first enteric coating is substantially insoluble in media with pH<3.

7. The pellets of claim 1, wherein the first enteric coating comprises a polymer selected from the group consisting of methacrylic acid copolymer, methyl methacrylate copolymer, and methacrylic acid-methyl methacrylate copolymer.

8. The pellets of claim 1, wherein the first sustained release coating of the plurality of third pellets comprises ammonio methacrylate copolymer.

9. The pellets of claim 2, wherein the plurality of second or fourth pellets comprises N-acetylcysteine, or a pharmaceutically acceptable salt or ester thereof, in between about 10% to about 90% by weight.

10. The pellets of claim 2, wherein the first polymer is selected from the group consisting of polyvinyl polypyrrolidone (PVPP), polyvinyl pyrrolidone (PVP), hydroxypropyl methylcellulose phthalate (HPMCP), hypromellose acetate succinate (HPMCAS), ethylcellulose, croscamolluse sodium (CMS-Na), Starch 1500, carboxymethyl cellulose sodium (CMC-Na), methacrylic acid copolymer, methyl methacrylate copolymer, a polymer comprising methacrylic acid-methyl methacrylate copolymer, EUDRAGIT® L100 and EUDRAGIT® L30D 55.

11. The pellets of claim 2, wherein the core of the plurality of second or fourth pellets further comprises a second polymer selected from the group consisting of cellulose, alkylated cellulose, and microcrystalline cellulose.

12. The pellets of claim 2, wherein the core the core of the plurality of second or fourth pellets further comprises a third polymer, selected from the group consisting of ethylene cellulose, hydroxypropylmethylcellulose, and polyvinyl pyrilodone.

13. The pellets of claim 2, wherein the core the core of the plurality of second or fourth pellets further comprises an antioxidant.

14. The pellets of claim 2, wherein the second enteric coating is substantially insoluble in media with pH<3.

15. The pellets of claim 2, wherein the second enteric coating comprises a polymer selected from the group consisting of methacrylic acid copolymer, methyl methacrylate copolymer, and methacrylic acid-methyl methacrylate copolymer.

16. The pellets of claim 1, wherein the second sustained release coating of the plurality of fourth pellets comprises ammonio methacrylate copolymer.

17. A pharmaceutical composition comprising a plurality of pellets selected from the group consisting of:
a) a plurality of the first pellets of claim 1 and a plurality of the second pellets of claim 2;
b) a plurality of the first pellets of claim 1 and a plurality of the third pellets of claim 1;
c) a plurality of the first pellets of claim 1 and a plurality of the fourth pellets of claim 2;
d) a plurality of the second pellets of claim 2 and a plurality of the third pellets of claim 1;
e) a plurality of the second pellets of claim 2 and a plurality of the fourth pellets of claim 2;
f) a plurality of the third pellets of claim 1 and a plurality of the fourth pellets of claim 2;
g) a plurality of the first pellets of claim 1, a plurality of the second pellets of claim 2, and a plurality of the third pellets of claim 1;
h) a plurality of the first pellets of claim 1, a plurality of the second pellets of claim 2, and a plurality of the fourth pellets of claim 2;
i) a plurality of the first pellets of claim 1, a plurality of the third pellets of claim 1, and a plurality of the fourth pellets of claim 2;
j) a plurality of the second pellets of claim 2, a plurality of the third pellets of claim 1, and a plurality of the fourth pellets of claim 2; and
k) a plurality of the first pellets of claim 1, a plurality of the second pellets of claim 2, a plurality of the third pellets of claim 1, and a plurality of the fourth pellets of claim 2.

18. A pharmaceutical formulation in a form selected from the group consisting of a capsule, a tablet, a mini-tablet, a suspension, a dose sipping straw, and a sachet, wherein the formulation comprises a plurality of pellets selected from the group consisting of:
a) a plurality of the first pellets of claim 1 and a plurality of the second pellets of claim 2;
b) a plurality of the first pellets of claim 1 and a plurality of the third pellets of claim 1;
c) a plurality of the first pellets of claim 1 and a plurality of the fourth pellets of claim 2;
d) a plurality of the second pellets of claim 2 and a plurality of the third pellets of claim 1;
e) a plurality of the second pellets of claim 2 and a plurality of the fourth pellets of claim 2;
f) a plurality of the third pellets of claim 1 and a plurality of the fourth pellets of claim 2;
g) a plurality of the first pellets of claim 1, a plurality of the second pellets of claim 2, and a plurality of the third pellets of claim 1;
h) a plurality of the first pellets of claim 1, a plurality of the second pellets of claim 2, and a plurality of the fourth pellets of claim 2;
i) a plurality of the first pellets of claim 1, a plurality of the third pellets of claim 1, and a plurality of the fourth pellets of claim 2;
j) a plurality of the second pellets of claim 2, a plurality of the third pellets of claim 1, and a plurality of the fourth pellets of claim 2; and
k) a plurality of the first pellets of claim 1, a plurality of the second pellets of claim 2, a plurality of the third pellets of claim 1, and a plurality of the fourth pellets of claim 2;
l) a plurality of the first pellets of claim 1;
m) a plurality of the second pellets of claim 2;
n) a plurality of the third pellets of claim 1; and
o) a plurality of the fourth pellets of claim 2.

19. The formulation of claim 18, wherein the form of the formulation is soluble in an aqueous solution having a pH less than 5, but is substantially insoluble in an aqueous solution having a pH greater than 7.
